(19) United States
(12) Patent Application Publication
(10) Pub. No.: US 2002/0195112 A1
(43) Pub. Date: Dec. 26, 2002

(54) Feeding device for feeding infants

(76) Inventor: Niva Shapira, Tel Aviv (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(21) Appl. No.: 10/190,842

(22) Filed: Jul. 9, 2002

Related U.S. Application Data
(63) Continuation of application No. 09/341,266, filed on Jul. 7, 1999.
(62) Division of application No. 09/887,028, filed on Jun. 25, 2001.

(51) Int. Cl. A61B 19/00

(52) U.S. Cl. 128/898

(57) ABSTRACT

A feeding device for feeding infants, includes a bottle open at a first end and closed at a second end opposite to the first end; and a nipple closing the first end of the bottle. The bottle is divided into at least a first compartment and a second compartment, each for containing a liquid, preferably a fore-milk equivalent liquid and a hind-milk equivalent liquid. The first compartment is in direct communication with the nipple to allow the infant to suck liquid directly therefrom. The second compartment is in communication with the nipple via the first compartment. In the described preferred embodiment, the bottle is constructed such that it first feeds to the infant the fore-milk equivalent liquid, and then feeds to the infant a mixture of the fore-milk equivalent liquid and the hind-milk equivalent liquid in a ratio in which the hind-milk equivalent liquid increases as the bottle is emptied.

### RELATED APPLICATIONS

The present application is a continuation of application Ser. No. 09/341,266, filed Jul. 7, 1999, and also relates to patent application Ser. No. 09/887,028, filed Jul. 7, 1999, which is a Division of that application.

### FIELD AND BACKGROUND OF THE INVENTION

The present invention relates to a method and feeding device for infant feeding and, more particularly, to a method for infant feeding with non-human milk which mimics breast fore- and hind-milk feeding, and to a feeding device for effecting the method.

Most researchers and authorities agree that under normal conditions exclusive breast-feeding provides substantially an adequate amount of most nutrients for infants for at least four months. Breast fed infants have advantages in terms of general health. They have a more effective immune system and are therefore less sensitive to infections, such as but not limited to, ears, lungs and intestinal infections, they achieve faster mental development and their body composition comprises less fat. Also, there are indications that breast-feeding achieves better future health of the child and the adult. For example, breast fed children are less prone to develop juvenile diabetes, heart diseases, obesity, etc. For further advantages of breast feeding the reader is referred to Am. J. Clin. Nutr. 40:635-646, 1984, which is incorporated by reference as if fully set forth herein.

It has been found that in the course of the breast feeding the infant is fed initially with milk having a smaller percentage of fat, which is known in the art as fore-milk, and thereafter with milk having a larger percentage of fat, which is known in the art as hind-milk. To this effect the reader is referred to J. of Ped. and Gastro. and Nutr. 18:474-477, 1994, which is incorporated by reference as if fully set forth herein.

In attempts to become as close as possible to breast feeding, formula producers invest a lot of effort in improving the composition of so called “humanised milk”. It has been found that formula fed infants tend to eat more calories and protein and to grow faster than breast fed infants. However, whereas in the past the rate of growth was perceived as an indication of good nutritional status, in recent years it became clear that facilitated growth is not necessarily advantageous to the present and future health of the child. To this effect the reader is referred to Am. J. Clin. Nutr. 58:152-161, 1993, and J. of Ped. and Gastro. and Nutr. 18:474-477, 1994, which are incorporated by reference as if fully set forth herein.

In view of the above research, it has become desirable to develop a method of feeding infants that provides a gradual increase of fat content which is similar to that of the breast feeding.

The advantage of the gradual method is assumed to be, inter alia, better compatibility between the infant's requirements and consumption. Infants who consume mostly the fore-milk, which is lower in fat content, will get more of the high carbohydrate low protein formula as the result of which they will get more protein which is spared (by carbohydrates) mainly for true growth of the infant, e.g., body length, without being satisfied for prolonged times and without over consumption of calories from fat. Bigger and stronger babies, who spend and require more energy, will get at the beginning more carbohydrate and protein, and later on more calories, but not as much protein, from extra suckling, as the fat content of the formula increases along the feeding session in, for example, a logarithmic or geometric manner.

There is thus a widely recognized need for, and it would be highly advantageous to have, a method for infant feeding by non-human milk which mimics breast fore- and hind-milk feeding, and a bottle for effecting the method.

### SUMMARY OF THE INVENTION

According to one aspect of the present invention there is provided a method for feeding with non-human milk, comprising the steps of (a) feeding the infant by fore-milk equivalent having a volume of, for example, 30-60% of a total meal and a percentage of fat of, for example, 2.5-3.5%; and (b) feeding the infant by hind-milk equivalent having a volume of, for example, 40-70% of the total meal and a percentage of fat of, for example, 3.7-5.5%.

According to still further features in the described preferred embodiments the feeding is performed either by feeding the fore-milk equivalent and the hind-milk equivalent one after another or by first feeding the fore-milk equivalent which is gradually admixed with the hind-milk equivalent.

According to still further features in the described preferred embodiments the percentage of the fore-milk equivalent in the total meal volume is 31-47% and the fat content therein is 2.8%-3.3%, and the percentage of the hind-milk equivalent in the total meal volume is 53-67% and the fat content therein is 4.5%-5%.

According to still further features in the described preferred embodiments the percentage of the fore-milk equivalent in the total meal is 40% and the fat content therein is 3%, and the percentage of the hind-milk equivalent in the total meal volume is 60% and the fat content therein is 4.8%.

According to still further features in one described preferred embodiment, the feeding is performed in two bottles, one being filled with the fore-milk equivalent liquid, and one being filled with the hind-milk equivalent liquid, the contents of the bottles being fed one after the other.

According to another aspect of the present invention, there is provided a feeding device for feeding infants, comprising: a bottle open at a first end and closed at a second end opposite to the first end; and a nipple closing the first end of the bottle; the bottle being divided into at least a first compartment and a second compartment, each for containing a liquid; the first compartment being in direct communication with the nipple to allow the infant to suck liquid directly therefrom; the second compartment being in liquid communication with the first compartment via a one-way valve permitting liquid flow from the second compartment to the first compartment, but not from the first compartment to the second compartment; whereby the first compartment may be supplied with a first liquid to be fed first to the infant directly via the nipple, and the second compartment may be supplied with a second liquid to be fed to the infant via the one-way valve, the first compartment, and the nipple.

In one described preferred embodiment, the first compartment is at the first end of the bottle adjacent to the nipple and is in direct fluid communication with the nipple; and in a second described preferred embodiment, the first compartment is at the second end of the bottle remote from the nipple and communicates with the nipple via a feed tube passing through the second compartment.

In both described preferred embodiments, the one-way valve is a one-way diaphragm valve.

According to further features in one described preferred embodiment, the first end of the bottle includes a one-way valve permitting liquid to be fed from the bottle via the nipple but not air to enter the bottle from the nipple; and wherein the second end of the bottle includes a one-way valve permitting air to enter the bottle to equalize the pressure within the bottle as liquid is being fed to the infant, but preventing liquid from exiting from the bottle through the one-way valve.

In a second described embodiment, the second end of the bottle is completely closed such that air enters via the nipple to equalize the pressure within the bottle as liquid is being fed to the infant.

According to further features in the described preferred embodiments, the bottle is constructed of at least two sections secured together, each section defining one of the compartments.

The feeding device described herein is particularly useful in the method referred to above for feeding an infant with non-human milk.

Therefore, according to another aspect of the present invention, there is provided a feeding device for feeding infants, comprising: a bottle open at a first end and closed at a second end opposite to the first end; and a nipple closing the first end of the bottle; the bottle being divided into at least a first compartment and a second compartment, each for containing a liquid; the first compartment containing a fore-milk equivalent liquid and being in direct communication with the nipple to allow the infant to suck the fore-milk equivalent liquid directly from the first compartment; the second compartment containing a hind-milk equivalent liquid and being in communication with the nipple via the first compartment.

According to further features in the described preferred embodiments, the second compartment is in liquid communication with the nipple via the first compartment and a one-way valve which permits of the hind-milk equivalent liquid flow from the second compartment to the first compartment, but not from the first compartment to the second compartment.

According to further features in one embodiment of this aspect of the invention, the bottle is constructed such that it first feeds to the infant the fore-milk equivalent liquid, and then feeds to the infant a mixture of the fore-milk equivalent liquid and the hind-milk equivalent liquid in a ratio in which the hind-milk equivalent liquid increases as the bottle is emptied.

The present invention thus successfully addresses the shortcomings of the presently known infant feeding techniques by providing a method and feeding device for infant feeding with non-human milk which mimics breast fore- and hind-milk feeding.

### BRIEF DESCRIPTION OF THE DRAWINGS

The invention herein described, by way of example only, with reference to the accompanying drawings, wherein:

FIG. 1 shows a front view of a bottle constructed according to the present invention; and

FIG. 2 shows a front view of another bottle constructed according to the present invention.

### DESCRIPTION OF THE PREFERRED EMBODIMENTS

The present invention is of a method for infant feeding by non-human milk and of a bottle for effecting the method, which can be used for mimicking breast fore- and hind-milk feeding. Specifically, the present invention can be used to gradually increase the fat content fed to the infant along a feeding session and thereby to mimic the natural case in which low fat fore milk followed by higher fat hind milk are fed to the infant.

The principles and operation of a method and bottle according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Thus, the present invention is of a method for gradual feeding of infants by non-human milk “infant formula”, wherein milk (fore-milk equivalent) having a volume of, for example, 30-60% of a total meal and a percentage of fat of, for example, 2.5-3.5%, and milk (hind-milk equivalent) having a volume of, for example, 40-70% of the total meal and a percentage of fat of, for example, 3.7-50.5% are fed to the infant. A meal may have a total volume of, for example, 30-300 ml, depending on infant's intake, age, weight, etc.

The term “milk equivalent” a used herein in the specification and in the claims section below refers to any human milk substitute used to feed infants. The substitute may be milk based or non-milk based (e.g., soy based). The terms “fore-milk equivalent” and “hind-milk equivalent” therefore refer to milk substitutes, as this term defined herein, which are similar or compatible at least in their fat content and/or other ingredients to human fore and hind milk, respectively.

According to the present invention feeding is performed either by feeding fore-milk equivalent and hind-milk equivalent one after another or alternatively by first feeding fore-milk equivalent which is gradually admixed with the hind-milk equivalent.

The percentage of the fore-milk equivalent in the total meal volume is preferably, 31-47%, advantageously 40%; and the fat content therein is preferably 2.8-3.3%, advantageously 3%.

The percentage of the hind-milk equivalent in the total meal volume is preferably 53-67%, advantageously 60%; and the fat content therein is preferably 4.5%-5%, advantageously 4.8%.

The several formulas to be fed by the method according to the present invention may be prepared, e.g., by dissolving in water a dry powder having the adequate formulation to achieve the correct content. The dissolving operation may be made either directly in the bottle or in a factory or the like where a large quantity of formulations is prepared and/or marketed as ready to feed liquid formula.

Milk base formulations according to the present invention may include the following ingredients: lactose, vegetable oils, skimmed milk powder, whey protein concentrate, Sodium, Calcium, Phosphorus, Potassium, Chloride, Iron, Magnesium, Taurine and Vitamins.

Soy base formulations according to the present invention may include the following ingredients: Glucose syrup, vegetable oils, soy protein isolate, Sucrose, Maltodextrine, Sodium, Calcium, Phosphorus, Potassium, chloride, Iron, Magnesium, Vitamins, Methionine, Taurine, Carnitine, and trace element.

Detailed composition of presently prefered soy and milk-based formulations of fore- and hind-milk equivalents are given in the Examples section hereinbelow.

The method according to the present invention may be performed by feeding with two separate bottles or a dual chamber bottle (as described, for example, in U.S. Pat. Nos. 5,593,052; 5,611,776; 5,419,445; 4,971,211, which are incorporated by reference as if fully set forth herein), i.e., by feeding fore-milk equivalent and hind-milk equivalent separately one after another, or with one combination bottle enabling the gradual admixing of hind-milk equivalent into fore-milk equivalent.

When the method is performed with two bottles (or a dual chamber bottle) each bottle (or chamber) is filled with one of the formulations. In this case at first the bottle (or chamber) containing the fore-milk equivalent is fed and thereafter the bottle (or chamber) containing the hind-milk equivalent. By this method the bottles utilised are conventional bottles for feeding infants.

Preferably, the method is performed by feeding with one combination bottle which is divided into two compartments, one being filled with fore-milk equivalent and the second one with hind-milk equivalent, the bottle being designed in such a manner enabling the infant to consume at first the fore-milk equivalent which is thereafter gradually admixed with the hind-milk equivalent.

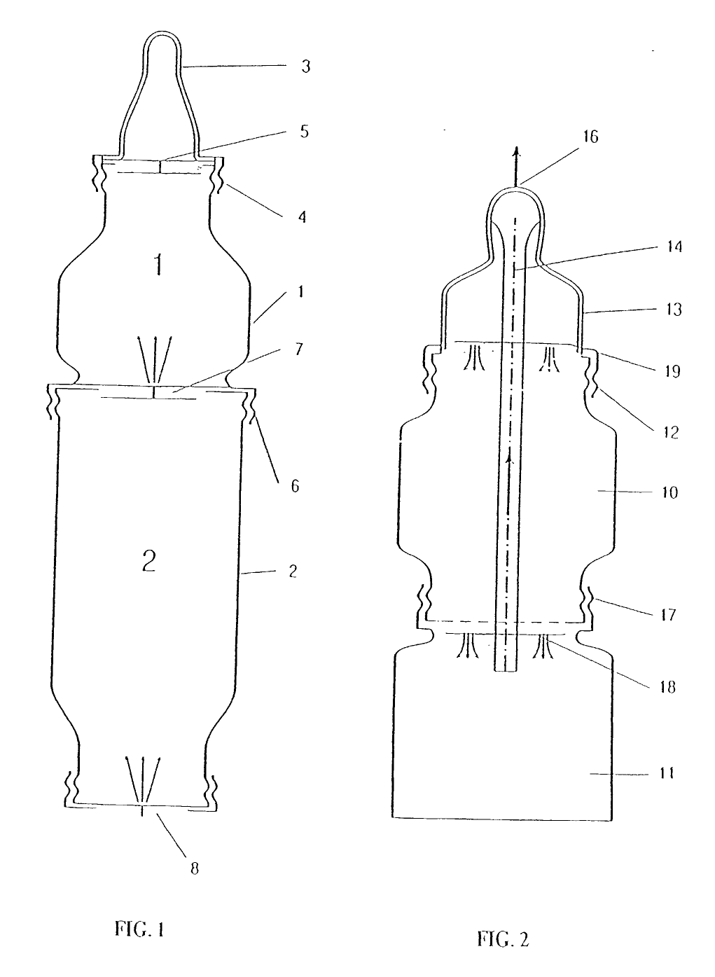

The bottle shown in FIG. 1 includes two compartments, compartment 1 for fore-milk equivalent and compartment 2 for hind-milk equivalent. Compartment 1 is closed at the front end by nipple 3 threaded thereon by threads 4 and by a non-return diaphragm valve 5. At its back end compartment 1 is connected to the front end of compartment 2 by thread 6 and by a non-return diaphragm valve 7. The back end of compartment 2 is provided with non-return diaphragm valve 8, which enables air to flow into compartment 2.

Diaphragm valves 8, 7 and 5 are one-way valves and enable the flow of air and/or milk in the same direction, i.e., from the back end of compartment 2 to compartment 1 and to nipple 3. Thus, the moment the infant starts to suckle, milk flows to its mouth, at the beginning only the fore-milk equivalent, and thereafter a gradual mixture of same with the hind-milk equivalent.

The bottle shown in FIG. 2 includes also two compartments, compartment 10 for hind-milk equivalent liquid, and compartment 11 for fore-milk equivalent liquid. Thread 12 connects the front end of compartment 10 to nipple 13. Nipple 13 is in fluid connection by feed tube 14 to compartment 11. Hole 16 in nipple 13 enables free flow of atmospheric air to compartment 10 via one or more one-way diaphragm valves 19. The back end of compartment 10 is connected to the front end of compartment 11 by threads 17 and one-way diaphragm valve 18.

The moment the infant starts to suckle via nipple 13 and feed tube 14, liquid flows from compartment 11; and as a result of the created vacuum, air flows through hole 16 to compartment 10. At the beginning, pure fore-milk equivalent is fed to the infant from compartment 11 through feed tube 14. Thereafter, a gradual mixture thereof with the hind-milk equivalent is fed from compartment 10 to compartment 11 and from there via feed tube 14 to the mouth of the infant.

It will thus be seen that both feeding devices illustrated in FIGS. 1 and 2 include a bottle open at a first end having the nipple (3, 13) and closed by an end wall at the opposite end. The bottle is divided into two compartments, namely a first compartment (1, 11) for the fore-milk equivalent liquid, and a second compartment (2, 10) for the hind-milk equivalent liquid.

In both embodiments, the first compartment is in direct communication with the nipple to allow the infant to suck liquid directly therefrom, and the second compartment is in liquid communication with the first compartment permitting liquid flow from the second compartment to the nipple via the first compartment. Thus, in FIG. 1, the first compartment 1 is in direct communication with the nipple 3, whereas the second compartment 2 communicates with the nipple 3 via the first compartment 1 and the one-way diaphragm valve 7. In FIG. 2, the first compartment 11, containing the fore-milk equivalent liquid, is in direct communication with the nipple 13 via the feed tube 14; and the second compartment 10 containing the hind-milk equivalent liquid communicates with the nipple 16 via the one-way diaphragm valve 18, the first compartment 11, and the feed tube 14.

In the FIG. 1 embodiment, the equalizing air enters the bottle via the one-way valve 8 in the closed end wall of the bottle; whereas in FIG. 2, the equalizing air enters the bottle via opening 16 in the nipple 13.

It will further be seen that in both embodiments, the bottle is constructed such that it first feeds to the infant the fore-milk equivalent liquid, and then feeds a mixture of the fore-milk equivalent liquid and the hind-milk equivalent liquid in a ratio in which the latter increases as the bottle is emptied.

Following are a number of examples of various formulations that may be used for both the fore-milk equivalent liquid to be included in compartment 1 of FIG. 1 or compartment 11 of FIG. 2, and of the hind-milk equivalent liquid to be included in compartment 2 of FIG. 1 and compartment 10 of FIG. 2:

### EXAMPLE 1

### Infant Formula I (Milk Base)

The formulation comprises the following ingredients: lactose, vegetable oils, skimmed milk powder, whey protein concentrate, Sodium, Calcium, Phosphorus, Potassium, Chloride, Iron, Magnesium, Taurine and Vitamins. A special formulation is given hereunder:

| | | | | |
|---|---|---|---|---|
| | | | Fore-Milk | Hind-Milk |
| | Powder | Regular | Eq. | Eq. |
| Unit | 100 g | 100 ml | 100 ml | 100 ml |

General Comp.

| | | | | | |
|---|---|---|---|---|---|
| Protein | gram | 11.1 | 1.5 | 1.5 | 1.5 |
| Fat | gram | 25.9 | 3.5 | 2.9 | 4.8 |
| Lactose | gram | 55.5 | 7.5 | 7.5 | 7.5 |
| Water | gram | 2.5 | — | — | — |
| Ash | gram | 2.06 | 0.27 | 0.27 | 0.27 |

Vitamins

| | | | | | |
|---|---|---|---|---|---|
| Vitamin A | I.U. | 1500 | 200 | 200 | 200 |
| Vitamin D | I.U. | 300 | 40 | 40 | 40 |
| Vitamin E | mg | 6 | 0.81 | 0.81 | 0.81 |
| Vitamin K | μg | 15 | 2.01 | 2.01 | 2.01 |
| Vitamin B1 | μg | 350 | 47.03 | 47.03 | 47.03 |
| Vitamin B2 | μg | 450 | 60 | 60 | 60 |
| Vitamin B6 | μg | 222 | 30 | 30 | 30 |
| Vitamin B12 | μg | 0.66 | 0.09 | 0.09 | 0.09 |
| Niacin | mg | 2 | 0.27 | 0.27 | 0.27 |
| Folic Acid | μg | 45 | 6 | 6 | 6 |
| Calcium | mg | 4.44 | 0.06 | 0.06 | 0.06 |
| Pantothenate | | | | | |
| Biotin | μg | 11 | 1.5 | 1.5 | 1.5 |
| Vitamin C | mg | 45 | 6.08 | 6.08 | 6.08 |

Minerals

| | | | | | |
|---|---|---|---|---|---|
| Calcium | mg | 326 | 44 | 44 | 44 |
| Phosphorus | mg | 219 | 29.6 | 29.6 | 29.6 |
| Magnesium | mg | 37 | 5 | 5 | 5 |
| Iron | mg | 7.4 | 1 | 1 | 1 |
| Sodium | mg | 120.7 | 16.3 | 16.3 | 16.3 |
| Potassium | mg | 373 | 50 | 50 | 50 |
| Ca/p ratio | — | 1.49 | 1.49 | 1.49 | 1.49 |

Amino Acid Profile

| | | | | | |
|---|---|---|---|---|---|
| Alanine | mg | 522 | 69.6 | 69.6 | 69.6 |
| Arginine | mg | 368 | 49.1 | 49.1 | 49.1 |
| Asparatic Acid | mg | 11.10 | 1.5 | 1.5 | 1.5 |
| Cystine | mg | 191 | 25.5 | 25.5 | 25.5 |
| Glutamic Acid | mg | 1423 | 189.7 | 189.7 | 189.7 |
| Glycine | mg | 244 | 32.5 | 32.5 | 32.5 |
| Histidine | mg | 262 | 34.9 | 34.9 | 34.9 |
| Isoleucine | mg | 761 | 101.5 | 101.5 | 101.5 |
| Leucine | mg | 12.20 | 1.62 | 1.62 | 1.62 |
| Lysine | mg | 10.00 | 1.3 | 1.3 | 1.3 |
| Methionine | mg | 270 | 36 | 36 | 36 |
| Phenylalanine | mg | 461 | 62.3 | 62.3 | 62.3 |
| Proline | mg | 962 | 128.3 | 128.3 | 128.3 |
| Serine | mg | 681 | 90.8 | 90.8 | 90.8 |
| Taurine | mg | 37 | 4.9 | 4.9 | 4.9 |
| Threonine | mg | 686 | 91.5 | 91.5 | 91.5 |
| Tryptophan | mg | 180 | 24 | 24 | 24 |
| Tyrosine | mg | 463 | 61.7 | 61.7 | 61.7 |
| Valine | mg | 775 | 103.3 | 103.3 | 103.3 |

Fatty Acid Profile

| | | | | | |
|---|---|---|---|---|---|
| Caprylic (C8) | % from fat | 2.6 | 2.6 | 2.6 | 2.6 |
| Capric (C10) | % | 2.1 | 2.1 | 2.1 | 2.1 |
| Lauric (C12) | % | 17.5 | 17.5 | 17.5 | 17.5 |
| Meristic (C14) | % | 6.7 | 6.7 | 6.7 | 6.7 |
| Palmitic (C16) | % | 11.2 | 11.2 | 11.2 | 11.2 |
| Stearic (C18) | % | 11.8 | 11.8 | 11.8 | 11.8 |
| Oleic (C18:1) | % | 37.0 | 37.0 | 37.0 | 37.0 |
| Linoleic (C18:2) | % | 10.0 | 10.0 | 10.0 | 10.0 |
| Linolenic (C18:3) | % | 1.2 | 1.2 | 1.2 | 1.2 |

The powder described herein is suitable for the preparation of the regular liquid formula. Similar powders having less or more fat content (see row 2) may be used for the preparation of the fore- and hind-milk equivalents, respectively.

### EXAMPLE 2

### Infant Formula II (Soy Base)

The formulation comprises the following ingredients: Glucose syrup, vegetable oils, soy protein isolate, Sucrose, Maltodextrine, Sodium, Calcium, Phosphorus, Potassium, chloride, Iron, Magnesium, Vitamins, Methionine, Taurine, Carnitine, and trace element. A special formulation is given hereunder:

| | | | | |
|---|---|---|---|---|
| | | | Fore-Milk | Hind-Milk |
| | Powder | Regular | Eq. | Eq. |
| Unit | 100 g | 100 ml | 100 ml | 100 ml |

General Comp.

| | | | | | |
|---|---|---|---|---|---|
| Protein | gram | 15 | 1.98 | 1.98 | 1.98 |
| Fat | gram | 27.54 | 3.64 | 3.0 | 4.7 |
| Carbohydrate | gram | 51.5 | 6.8 | 6.8 | 6.8 |
| Linoleic Acid | gram | 4.5 | 0.6 | 0.6 | 0.6 |

Vitamins

| | | | | | |
|---|---|---|---|---|---|
| Vitamin A | I.U. | 1500 | 198 | 198 | 198 |
| Vitamin D | I.U. | 300 | 39.7 | 39.7 | 39.7 |
| Vitamin E | I.U. | 10 | 1.32 | 1.32 | 1.32 |
| Vitamin C | mg | 65 | 8.6 | 8.6 | 8.6 |
| Vitamin K | μg | 77 | 10.2 | 10.2 | 10.2 |
| Vitamin B1 | μg | 345 | 45.6 | 45.6 | 45.6 |
| Vitamin B2 | μg | 445 | 58.9 | 58.9 | 58.9 |
| Vitamin B6 | μg | 327 | 43.3 | 43.3 | 43.3 |
| Vitamin B12 | μg | 1.5 | 0.2 | 0.2 | 0.2 |
| Niacin | mg | 7 | 0.93 | 0.93 | 0.93 |
| Folic Acid | μg | 76 | 10 | 10 | 10 |
| Pantothenic Acid | μg | 4.5 | 0.6 | 0.6 | 0.6 |
| Biotin | μg | 25 | 3.3 | 3.3 | 3.3 |
| Choline | mg | 58 | 7.7 | 7.7 | 7.7 |

Minerals

| | | | | | |
|---|---|---|---|---|---|
| Calcium | mg | 500 | 66.2 | 66.2 | 66.2 |
| Phosphorus | mg | 300 | 39.7 | 39.7 | 39.7 |
| Magnesium | mg | 45 | 6 | 6 | 6 |
| Iron | mg | 9.2 | 1.2 | 1.2 | 1.2 |
| Zinc | Mg | 4 | 0.53 | 0.53 | 0.53 |
| Manganese | μg | 150 | 19.8 | 19.8 | 19.8 |
| Copper | μg | 400 | 53 | 53 | 53 |
| Iodine | μg | 77 | 10.2 | 10.2 | 10.2 |
| Sodium | mg | 200 | 26.5 | 26.5 | 26.5 |
| Potassium | mg | 546 | 72.2 | 72.2 | 72.2 |
| Chloride | mg | 400 | 53 | 53 | 53 |
| Inositol | mg | 25 | 3.3 | 3.3 | 3.3 |
| Carnitine | mg | 10 | 1.3 | 1.3 | 1.3 |
| Ca/P ratio | mg | 1.67 | 1.67 | 1.67 | 1.67 |

Amino Acid Profile

| | | | | | |
|---|---|---|---|---|---|
| Alanine | mg | 640 | 85.3 | 85.3 | 85.3 |
| Arginine | mg | 497 | 6.5 | 6.5 | 6.5 |
| Aspartic Acid | mg | 1385 | 184.7 | 184.7 | 184.7 |
| Cystine | mg | 242 | 32.3 | 32.3 | 32.3 |
| Glutamic Acid | mg | 3065 | 408.7 | 408.7 | 408.7 |
| Glycine | mg | 300 | 40 | 40 | 40 |
| Histidine | mg | 382 | 50.9 | 50.9 | 50.9 |
| Isoleucine | mg | 893 | 119.1 | 119.1 | 119.1 |
| Leucine | mg | 1600 | 213.3 | 213.3 | 213.3 |
| Lysine | mg | 1360 | 181.3 | 181.3 | 181.3 |
| Methionine | mg | 406 | 54.1 | 54.1 | 54.1 |
| Phenylalanine | mg | 650 | 86.7 | 86.7 | 86.7 |
| Proline | mg | 1113 | 148.4 | 148.4 | 148.4 |
| Serine | mg | 737 | 98.3 | 98.3 | 98.3 |
| Taurine | mg | 51 | 6.8 | 6.8 | 6.8 |
| Threonine | mg | 460 | 61.3 | 61.3 | 61.3 |
| Tyrosine | mg | 621 | 82.8 | 82.8 | 82.8 |
| Valine | mg | 947 | 126.3 | 126.3 | 126.3 |

Fatty Acid Profile

| | | | | | |
|---|---|---|---|---|---|
| Caprylic (C8) | % from fat | 2.6 | 2.6 | 2.6 | 2.6 |
| Capric (C10) | % | 2.1 | 2.1 | 2.1 | 2.1 |
| Lauric (C12) | % | 17.5 | 17.5 | 17.5 | 17.5 |
| Meristic (C14) | % | 6.7 | 6.7 | 6.7 | 6.7 |
| Palmitic (C16) | % | 11.2 | 11.2 | 11.2 | 11.2 |
| Stearic (C18) | % | 11.8 | 11.8 | 11.8 | 11.8 |
| Oleic (C18:1) | % | 37.0 | 37.0 | 37.0 | 37.0 |
| Linoleic (C18:2) | % | 10.0 | 10.0 | 10.0 | 10.0 |
| Linolenic (C18:3) | % | 1.2 | 1.2 | 1.2 | 1.2 |

The powder described herein is suitable for the preparation of the regular liquid formula. Similar powders having less or more fat content (see row 2) may be used for the preparation of the fore- and hind-milk equivalents, respectively.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A feeding device for feeding infants, comprising:
a bottle open at a first end and closed at a second end opposite to said first end;
and a nipple closing said first end of the bottle;
said bottle being divided into at least a first compartment and a second compartment, each for containing a liquid;
said first compartment being in direct communication with said nipple to allow the infant to suck liquid directly therefrom;
said second compartment being in liquid communication with said first compartment via a one-way valve permitting liquid flow from the second compartment to the first compartment, but not from the first compartment to the second compartment;
whereby said first compartment may be supplied with a first liquid to be fed first to the infant directly via said nipple, and the second compartment may be supplied with a second liquid to be fed to the infant via said one-way valve, said first compartment, and said nipple.

2. The feeding device according to claim 1, wherein said first compartment is at said first end of the bottle adjacent to said nipple and is in direct fluid communication with said nipple.

3. The feeding device according to claim 1, wherein said first compartment is at said second end of the bottle remote from said nipple and communicates with said nipple via a feed tube passing through said second compartment.

4. The feeding device according to claim 1, wherein said one-way valve is a one-way diaphragm valve.

5. The feeding device according to claim 1, wherein said first end of the bottle includes a one-way valve permitting liquid to be fed from the bottle via the nipple but not air to enter the bottle from the nipple; and wherein said second end of the bottle includes a one-way valve permitting air to enter the bottle to equalize the pressure within the bottle as liquid is being fed to the infant, but preventing liquid from exiting from the bottle through said one-way valve.

6. The feeding device according to claim 1, wherein said second end of the bottle is completely closed such that air enters via said nipple to equalize the pressure within the bottle as liquid is being fed to the infant.

7. The feeding device according to claim 1, wherein said bottle is constructed of at least two sections secured together, each section defining one of said compartments.

8. The feeding device according to claim 7, wherein said two sections are secured together by threads.

9. The feeding device according to claim 1, wherein said first compartment contains a quantity of a fore-milk equivalent liquid to be fed first to the infant directly via said nipple; and said second compartment contains a quantity of a hind-milk equivalent liquid to be fed subsequently to the infant via said one-way valve, said first compartment, and said nipple.

10. The feeding device according to claim 9, wherein said first compartment is at said first end of the bottle adjacent to said nipple and is in direct fluid communication with said nipple.

11. The feeding device according to claim 9, wherein said first compartment is at said second end of the bottle remote from said nipple and communicates with said nipple via a feed tube passing through said second compartment.

12. A feeding device for feeding infants, comprising:
a bottle open at a first end and closed at a second end opposite to said first end;
and a nipple closing said first end of the bottle;
said bottle being divided into at least a first compartment and a second compartment, each for containing a liquid;
said first compartment containing a fore-milk equivalent liquid and being in direct communication with said nipple to allow the infant to suck said fore-milk equivalent liquid directly from said first compartment;
said second compartment containing a hind-milk equivalent liquid and being in communication with said nipple via said first compartment.

13. The feeding device according to claim 12, wherein said first compartment is at said first end of the bottle adjacent to said nipple and is in direct fluid communication with said nipple.

14. The feeding device according to claim 12, wherein said first compartment is at said second end of the bottle remote from said nipple and communicates with said nipple via a feed tube passing through said second compartment.

15. The feeding device according to claim 12, wherein said second compartment is in liquid communication with said nipple via said first compartment and a one-way valve which permits flow of said hind-milk equivalent liquid from the second compartment to the first compartment, but not from the first compartment to the second compartment.

16. The feeding device according to claim 15, wherein said one-way valve is a one-way diaphragm valve.

17. The feeding device according to claim 15, wherein said first end of the bottle includes a one-way valve permitting said fore-milk equivalent liquid to be fed via the nipple but does not permit air to enter the bottle via the nipple; and wherein said second end of the bottle includes a one-way valve permitting air to enter the bottle to equalize the pressure within the bottle as hind-milk equivalent liquid is being fed to the infant, but preventing said latter liquid from exiting from the bottle.

18. The feeding device according to claim 15, wherein said second end of the bottle is completely closed such that air enters via said nipple to equalize the pressure within the bottle as liquid is being fed to the infant.

19. The feeding device according to claim 12, wherein said bottle is constructed of at least two sections secured together, each section defining one of said compartments.

20. The feeding device according to claim 12, wherein said bottle is constructed such that it first feeds to the infant the fore-milk equivalent liquid, and then feeds to the infant a mixture of the fore-milk equivalent liquid and the hind-milk equivalent liquid in a ratio in which the hind-milk equivalent liquid increases as the bottle is emptied.
